(19) United States
(12) Patent Application Publication
(10) Pub. No.: US 2009/0064399 A1
(43) Pub. Date: Mar. 12, 2009

(54) Frame-Reduced Swim Goggles with Angle-Free Lens

(76) Inventor: Terry CHOU, Talnan City (TW)

Correspondence Address: Dr. BANGER SHIA;Int''l Patent Office of Bang Shia, 102 Lindencrest Ct, Sugar Land, TX, 77479-5201, US

(21) Appl. No.: 11/852,313

(22) Filed: Sep. 9, 2007

(51) Int. Cl. A61F 9/02

(52) U.S. Cl. 2/442; 2/426; 2/445

(57) ABSTRACT

The present invention discloses a pair of holistically designed swimming goggles, including two lens, two lens-receiving pads, and one or more head securing straps. The specialized lens includes a lens-pad engaging area, a lens-strap engaging piece, and a smooth curve transition between its front and lateral view without having an angle formed therein. Each of lens-receiving pads includes a loop-like lens-receiver, a pad extension, an eyes-goggles interface and a nose bridge. The secure engagement between lens and lens-receiving pads is achieved by having a substantially increased contact area between them.

### BACKGROUND OF THE INVENTION

1. Field of the Invention

The present invention relates to a pair of swimming goggles of simplified and yet sophisticated design. In particular, the present invention relates to a combination of two pieces of specialized lens, two matching lens-receiving pads and one or more securing straps, which has a broadened view, more resistance to water flow, and also substantial durability.

2. Description of the Related Art

Most conventional swim goggles include in general four main components: the lens, the frame and the padding, plus the securing head strap. In conventional goggles, a frame provides both a securing and a connecting function for an assembly of lens and padding. Three related conventional goggles are discussed below, and illustrated in FIGS. 7 and 8 for the first prior art, in FIGS. 9 and 10 for a second prior art, and in FIG. 11 for a third prior art.

FIGS. 7 and 8 of the drawings illustrates a pair of conventional swimming goggles, comprising two lens 10′, a nose-bridge 11′, two padding members 12′, and a head securing strap 13′. Detailed description of this prior art is given below.

Referring to FIGS. 7 and 8, the two lens 10′ are made of transparent material; each of lens 10′ comprises a front-view 101′, a side-view 102′, and an angular-view 103′ in between the front-view 101′ and the side-view 102′. The protruding flange 104′ is formed at the rear of each of lens 10′, extending outwards. A head strap connecting piece 105′ is disposed at one side of each of lens 10′ near the temple position of a goggle wearer for engaging with the head securing strap 13′. On the other side of each of lens 10′, corresponding to either side of the nose position is located a nose-bridge receiver 106′. In a similar way, the two ends of the nose-bridge 11′ connect to the two nose-bridge receivers 106′. The two padding members 12′ are made of soft materials, of which the front is formed a lens-engaging compartment 121′ for receiving the protruding flange 104′ (of the lens 10′). At the rear of each padding member 12′ neighboring the lens-engaging compartment 121′ is disposed an extension 122′ (of the lens-engaging compartment), while an eye-goggles interface is configured as a further extension of the extension 122′ (of the lens-engaging compartment). The head strap connecting piece 13′ engages with the head strap connecting piece 105′.

Referring to FIGS. 9 and 10, which illustrate a pair of conventional swimming goggles, comprising two lens 20′, two frames 21′, two padding members 22′, a central bridge 23′, and a head strap 24′. The two lens 20′ are transparent flat bodies; the two frames 21′, two padding members 22′, and a central bridge 23′ are made of soft material and produced as one single piece in manufacturing. The head strap 24′ engages with the two frame 21′, within each of which a frame-receiving compartment 211′ is formed to engage with the frames 21′.

The disadvantages associated with the conventional goggles shown in FIG. 10 arises from the fact that since frames 21′ are not visible, the visible part through the transparent lens 20′ is represented by an arc area with a narrow angle c. In addition, the frame-receiving compartment 211′ has a compartment wall 212′ for enclosing the lens 20′ within; the very presence of such a compartment wall 212′ further blocks a goggle wearer' view.

A third related prior art is shown in FIG. 11. The transparent lens 30′ are assembled together with a padding member 31′. An angular view 303′ (of the lens 30′) is disposed in between the front view 301′ and the lateral view 302′. At the rear of the lens 30′ is formed a receiving compartment 304′, from which extends a protruding rim 305′. The padding member 31′ is made of soft material and includes a pad-lens engaging part 311′, a padding extension 312′, an eye-goggle interface 313.′ The pad-lens engaging part 311′ is then assembled together with the receiving compartment 304′ and the protruding rim 305′.

Referring to FIG. 8-11, some disadvantages associated with these three prior arts are summarized as follows:
(1) In terms of utility practicability: Objects in front of lens in the above-discussed prior arts are presumably visible via an arc area with a see-through angle a. Nevertheless, due to the presence of an angular-view (in between the front and the lateral view of lens), the otherwise see-through angle a is decreased to a smaller angle b; likewise, the corresponding arc area is accordingly decreased, too.
(2) In terms of dynamic fluids: The angular-view of lens adversely affects the water resistance of the goggles when moving in water.
(3) In terms of structure durability: The loosen engagement between the padding members and the lens cause them to fall apart very easily.
(4) In terms of aesthetic design: The engaging between the padding members and lens is visibly exposed outsides, defying the spirit of an ensemble design.

### SUMMARY OF THE INVENTION

The goal of the present invention is to provide a pair of swimming goggles with improved assembling convenience, less water resistance, a broadened view, and a streamline ensemble appealing look at a reduced manufacturing cost, without affecting its utility durability.

The present invention distinguishes itself from related prior arts in that only three main components are utilized: lens, padding, and the head securing strap(s). The structure of the present invention is substantially simplified and, at the same time, the all-time disadvantage of blocking a swimmer's lateral view by a no-transparent frame or angular lens is avoided.

A pair of swimming goggles in accordance with an aspect of the invention comprises two lens, two pads, and one or more head securing straps. The lens are made of transparent material, each of which includes an up-front view (right in front of eyes), a lateral view, and a smooth angle-less curve surface configured in between the up-front view and the lateral view; the design of such a smooth transition curve between the up-front view and the lateral view substantially increases the range of a view. Along the perimeter of the rear surface of lens is formed a lens-pad engaging rim; while a lens-strap engaging window is formed at each end of lens, neighboring the lateral view.

Each pad is made of soft material and configured with a lens-receiving compartment to engage with its corresponding piece of lens. A patentably distinguished characteristic of the present invention also include the lens-receiving compartment of the lens, which is configured not to enclose or block any part of the lens (to which the compartment is engaged). Gluing material may be applied to secure the engagement between the lens and the padding; or alternatively, the lens and the padding are made together as a single piece in manufacture to enhance its appealing look.

Other objects, advantages, and novel features of the invention will become more apparent from the following detailed description when taken in conjunction with the accompanying drawings.

### BRIEF DESCRIPTIONS OF THE DRAWINGS

FIG. 1 is an exploded perspective view of the 1^{st} embodiment of a pair of swimming goggles in accordance with the present invention.

FIG. 2 is a partial assembly view of the goggles shown in FIG. 1.

FIG. 3 illustrates the pair of swimming goggle in FIG. 2 along the vertical A-A cross-sectional view.

FIG. 4 illustrates the pair of swimming goggles in FIG. 2 along the horizontal B-B cross-sectional view.

FIG. 5 is an assembly perspective view of the 2^{nd} embodiment of a pair of lens and padding in accordance with the present invention.

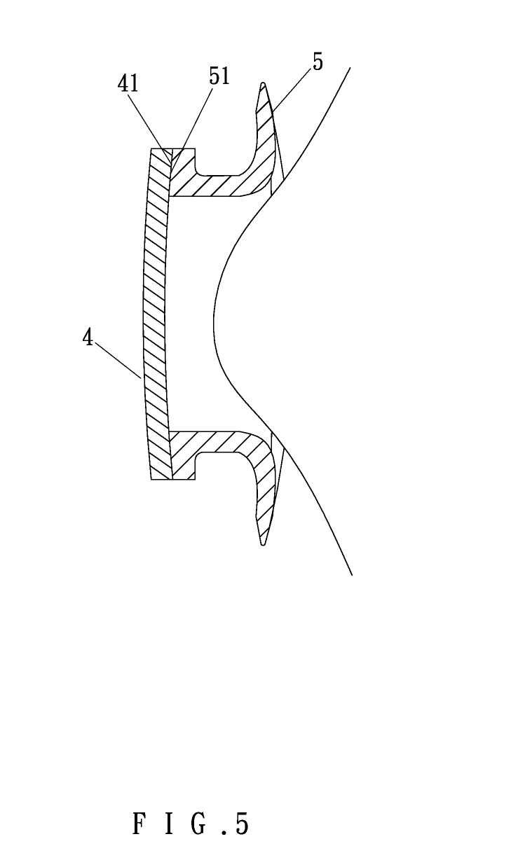

FIG. 6 is an assembly perspective view of the 3^{rd} embodiment of a pair of lens and padding in accordance with the present invention.

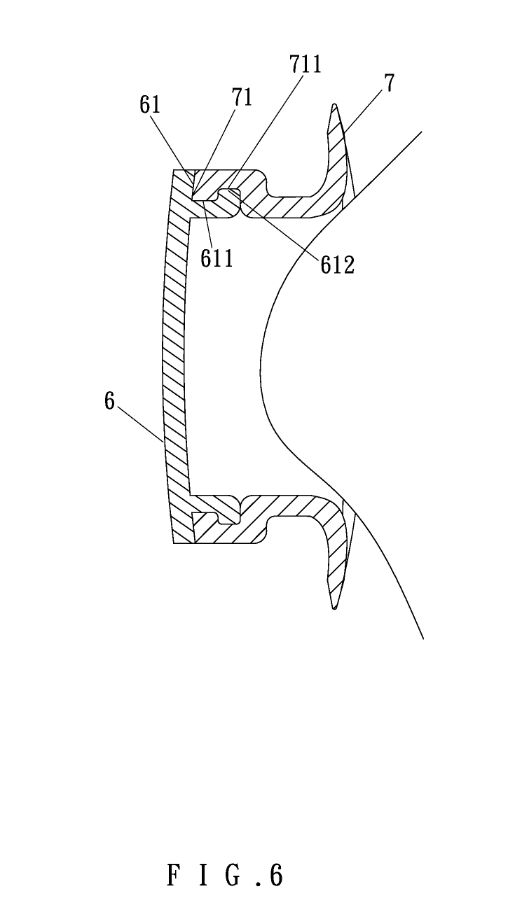

FIG. 7 is an exploded perspective view of the 1^{st} pair of conventional swimming goggles.

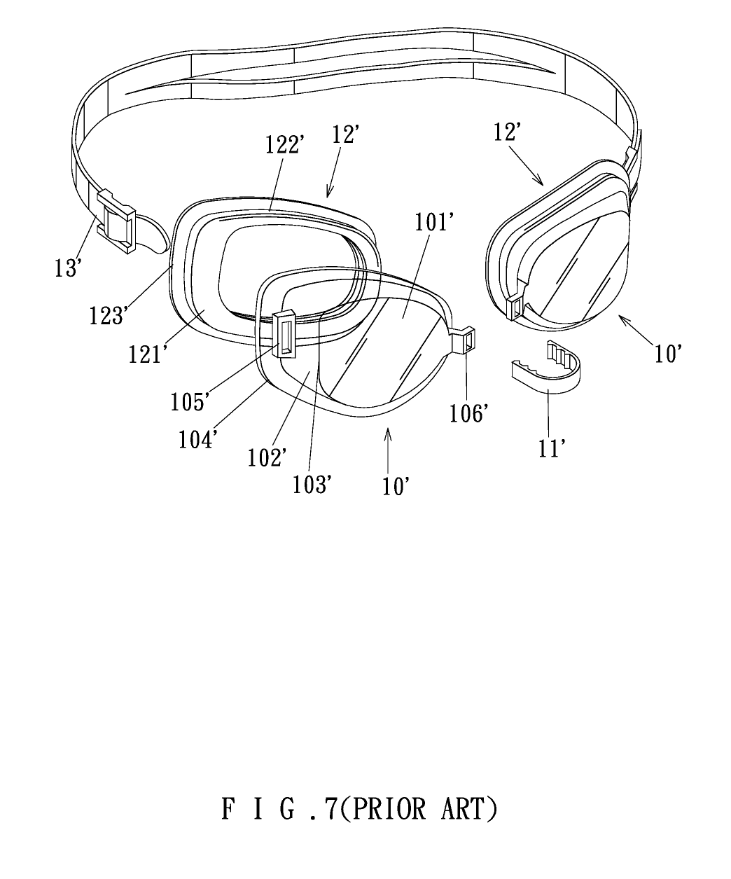

FIG. 8 is an assembly perspective view of the 1^{st} pair of conventional swimming goggles.

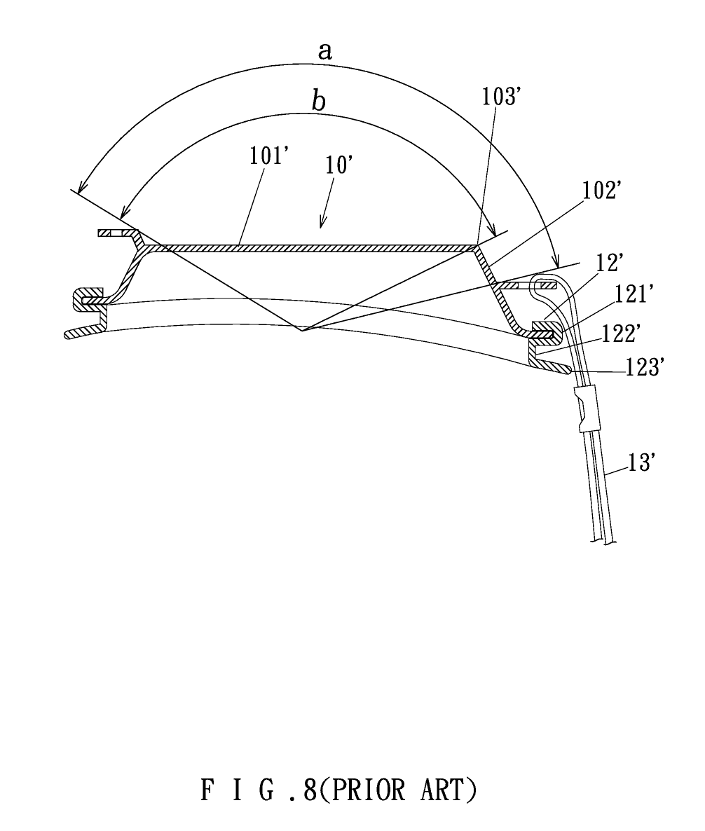

FIG. 9 is an exploded perspective view of the 2^{nd} pair of conventional swimming goggles.

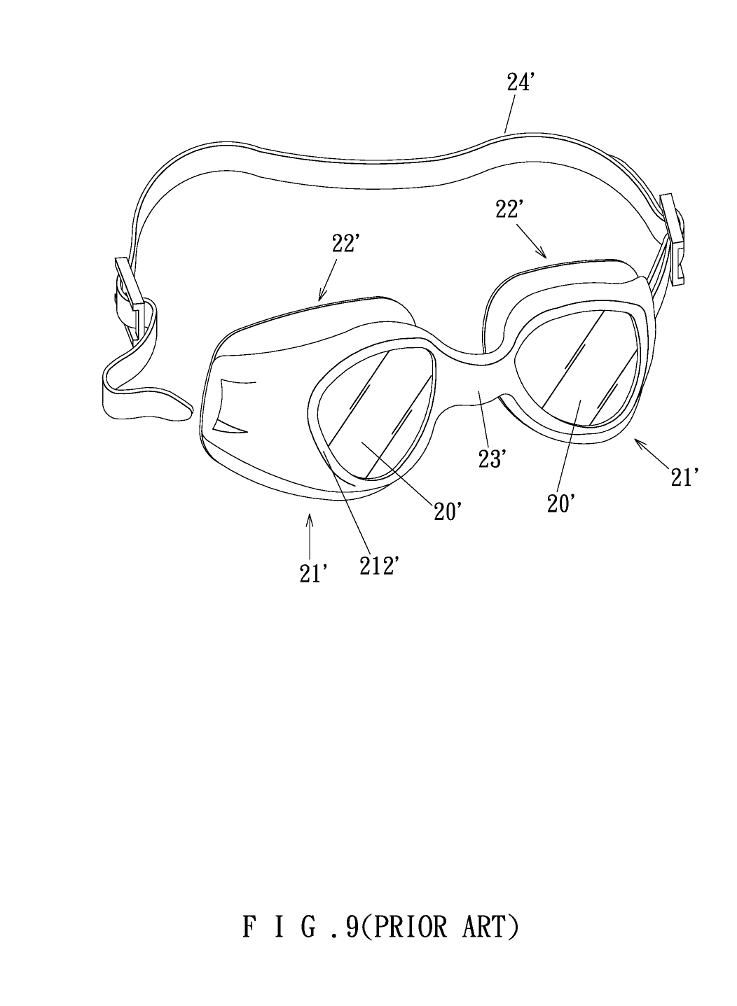

FIG. 10 is an assembly perspective view of the 2^{nd} pair of conventional swimming goggles.

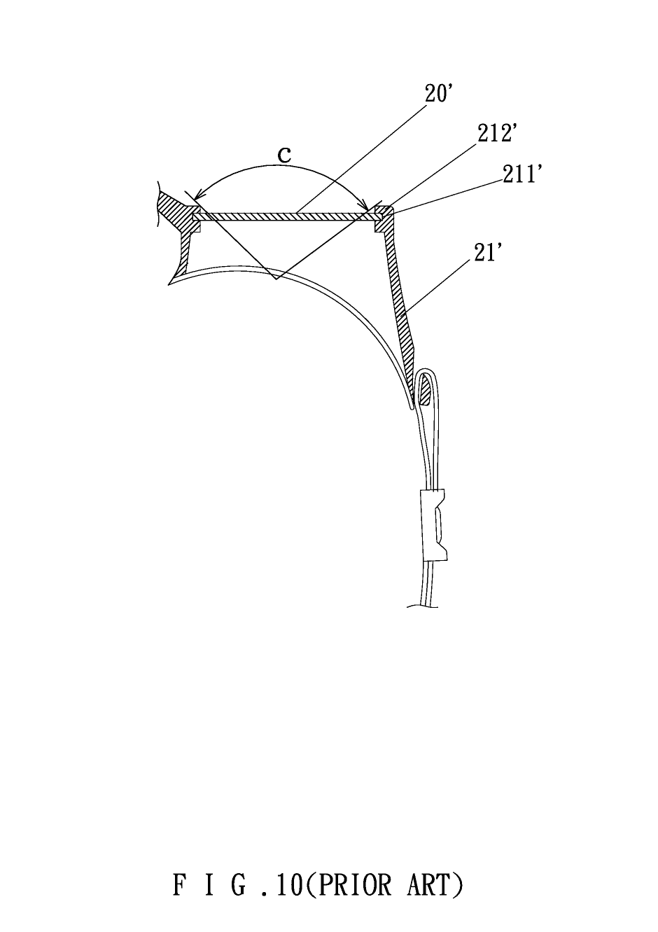

FIG. 11 is an assembly perspective view of the 3^{rd} pair of conventional swimming goggles.

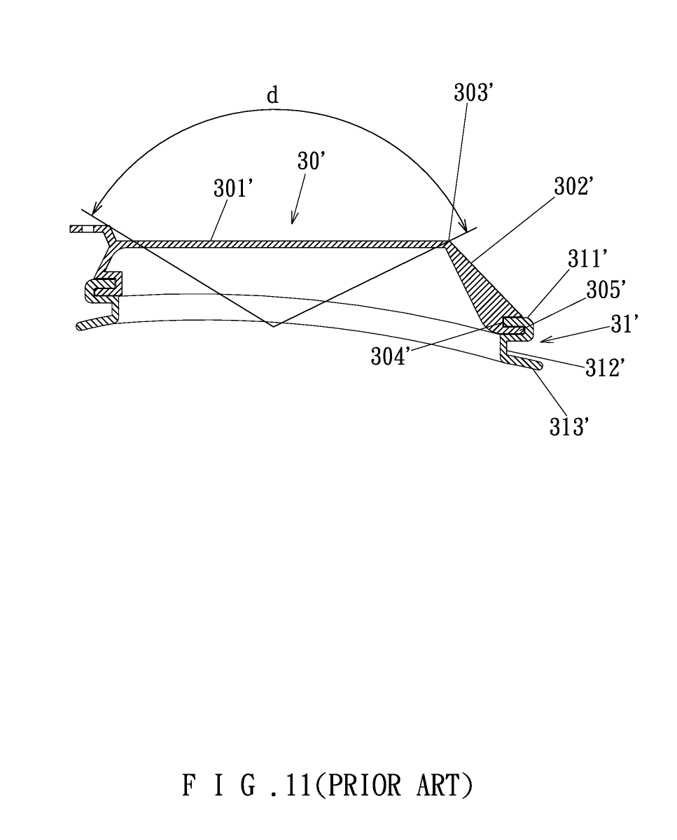

### DETAILED DESCRIPTIONS OF THE DRAWINGS

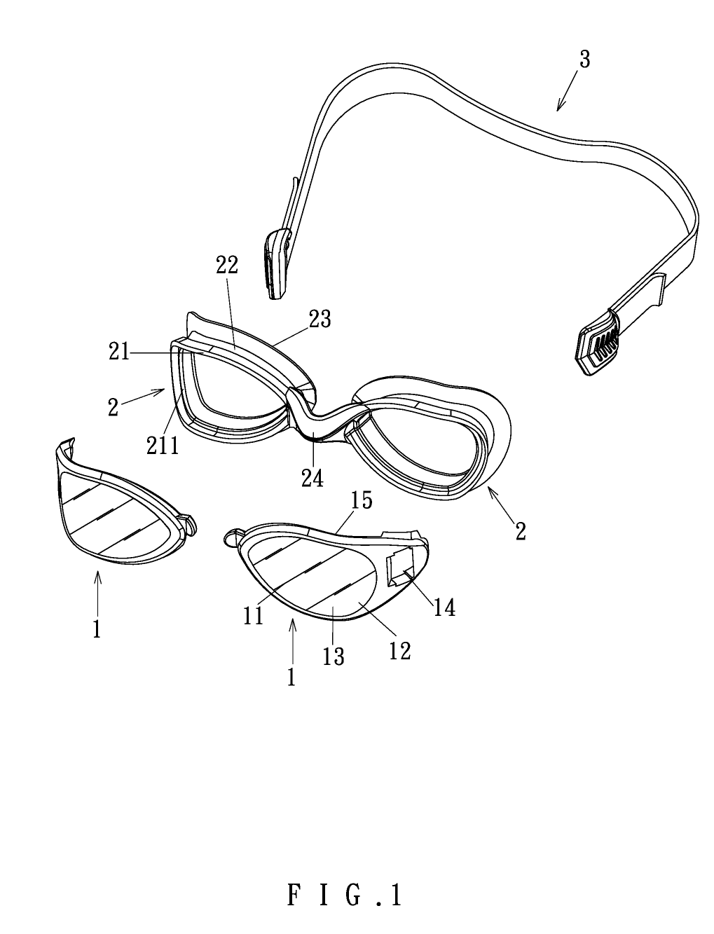

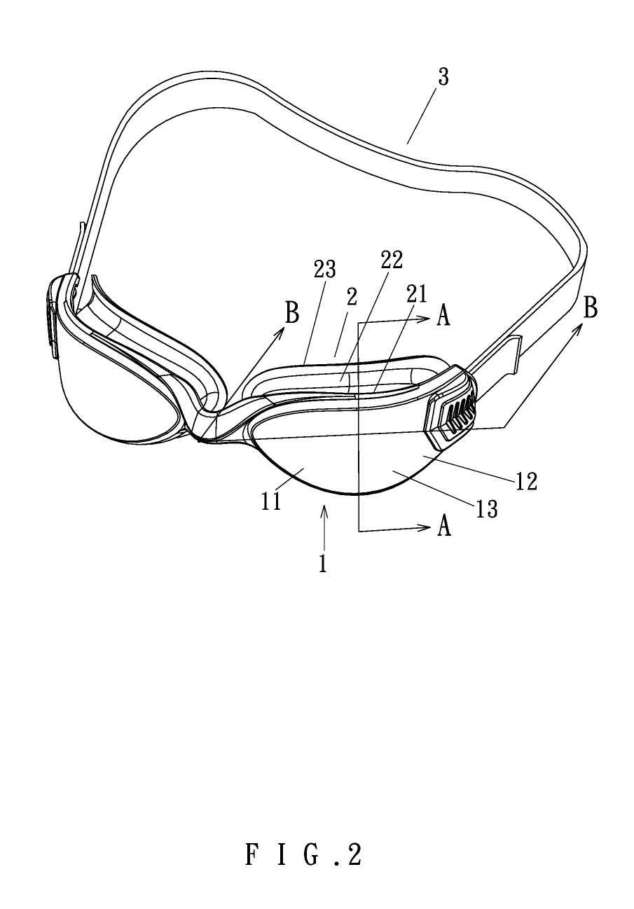

Referring to FIGS. 1 to 3, a piece of swim goggles in accordance with the present invention comprises mainly two pieces of lens 1, two lens-receiving pads 2, and one or more head securing straps 3. Detail descriptions are given below for each component.

(1) Lens: Lens 1 further includes five sections:
(a) the front view 11,
(b) the lateral view 12,
(c) a smooth transition curve surface 13 between the front view 11 and the lateral view 12,
(d) a strap engaging piece 14, and
(e) a lens-padding engaging area 15, from which a rear protruding flange 151 extends outwards.

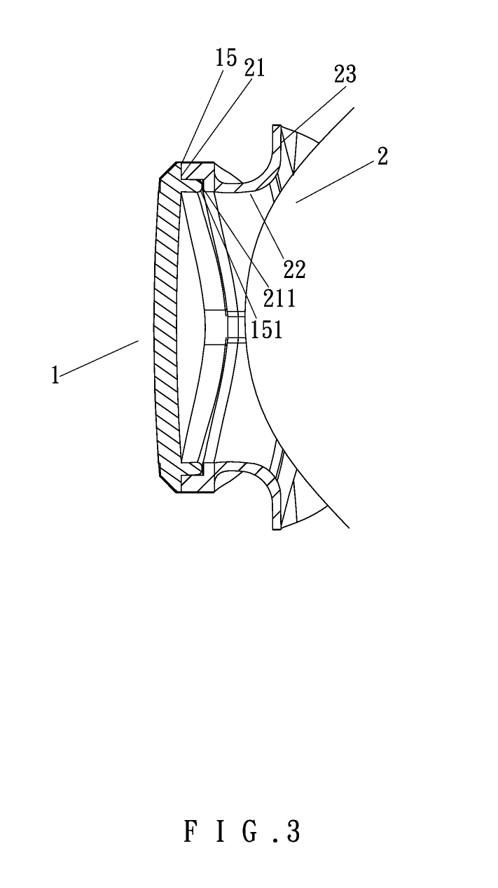

(2) Lens-receiving pads: The lens-receiving pads 2 are made of soft material and include:
(a) two loop-like lens receivers 21, formed along the inner perimeter of the pair of lens. The loop-like lens receiver 21 is designed to fit each of lens 1 so that the combination of the two comfortably and securely sit around the eyes of a swimmer; loop-like lens receiver 21 is especially configured not to enclose or block any part of the lens by extending outwards in a direction vertical to the direction or phase of the lens; a pad-lens contact wall 211, formed to correspond to the physical configuration of the above-discussed rear protruding flange 151 to allow the engagement between the loop-like lens receiver 21 and the lens-padding engaging area 15. As shown in FIG. 3, the contact area and hence the engagement between the pad-lens contact wall 211 and the rear protruding flange 151 substantially increases. The loop-like lens-receiver 21 can be engaged with the lens-padding engaging area 15 (of the lens 1) by gluing or by being manufactured together in production. As a result, more durable securing between the lens and the pads is achieved by having a larger contact area via the engagement between the rear protruding flange 151 and the pad-lens contact wall 211;
(b) the pad extension 22, which further extends along the loop-like lens-receiver 21;
(c) the nose bridge 24; and
(d) the eyes-goggles interface 23, formed to neighbor the pad extension 22.

(3) the head securing strap 3, each of which connects to the lens 1 via its corresponding strap engaging piece 14 to fit a swimmer's forehead. Alternatively, the head securing strap 3 can also interlock with the strap engaging piece 14 by some connecting piece (not shown in the figures).

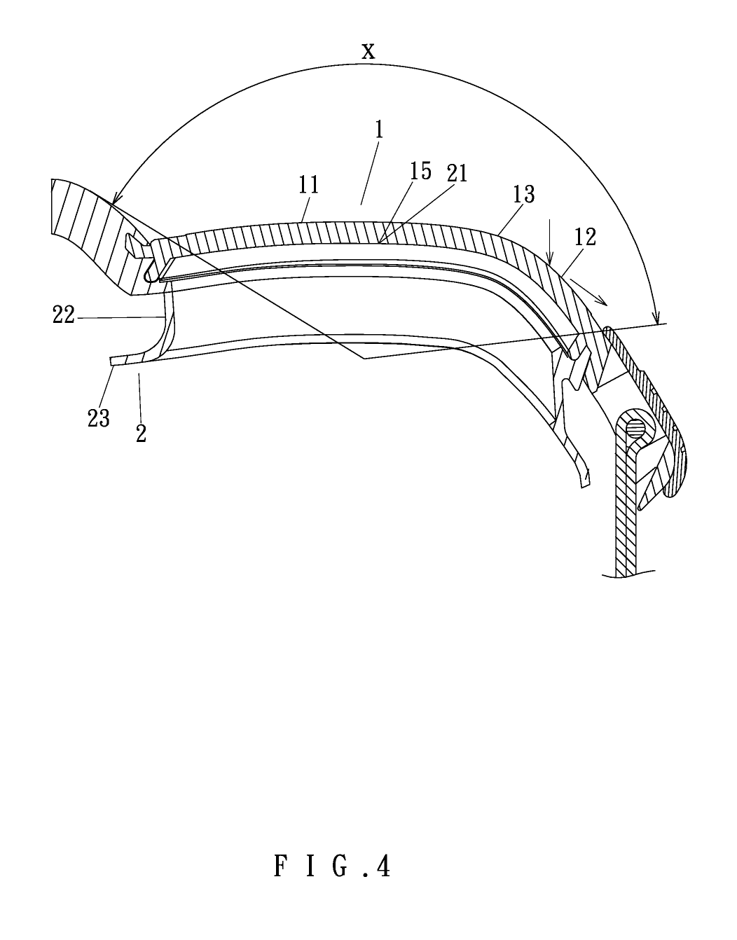

Please further refer to FIGS. 3 and 4 for a summary of the distinguishing characteristics of the present invention:
(1) Due to the specially designed angle-less and frame-less lens, the area of visible view substantially increases, as embodied by the cross-sectional view of an arc area with an angle of x in the figures. The arrow in the figure indicates the smooth transition curve surface 13, which also reduces the resistance of flowing water against the forward moving of goggles under water.
(2) The interface between the lens and the receiving pads is so designed that no part of the pad will ever block or enclose any part of the lens, and at the same time, maintaining the utility durability by having a substantially increased contact or engagement area between the lens and pads.

Although specific embodiments have been illustrated and described, numerous modifications and variations are still possible without departing from the essence of the invention. The scope of the invention is limited by the accompanying claims.

What is claimed is:

1. A pair of swimming goggles, comprising:
(I) two lens, made of transparent material, each comprising:
(1) a front view,
(2) a lateral view,
(3) a front-lateral curve of a smooth transition between said front view and said lateral view without an angle formed therein,
(4) a lens-pad engaging area, along the perimeter of the rear surface of each said lens, and
(5) a lens-strap engaging piece, facing away from a goggle wearer;
(II) two lens-receiving pads, made of soft materials, each comprising:
(1) a loop-like lens-receiver, formed along the outer perimeter of said lens-receiving pad and configured to interlock with said lens-pad engaging but not to block any part of each said lens,
(2) a pad extension, which extends from said loop-like lens-receiver,
(3) an eyes-goggles interface, neighboring said pad extension, and
(4) a nose bridge;
(III) a head securing strap assembly, the two ends of which connects respectively to those of said lens.

2. The pair of swimming goggles as claimed in claim 1, wherein the engagement between said lens-pad engaging area and said loop-like lens-receiver is made by gluing.

3. The pair of swimming goggles as claimed in claim 1, wherein the engagement between said lens-pad engaging area and said loop-like lens-receiver is fixed in manufacturing.

4. The pair of swimming goggles as claimed in claim 1, wherein a rear protruding flange extends outwards from said lens-pad engaging area, and a matching pad-lens contact wall is physically formed to correspond to said rear protruding flange for engagement.

5. The pair of swimming goggles as claimed in claim 1, wherein each of said lens comprising a loop-like concave rim and a protruding rim; each of said lens-receiving pad comprising a pad-lens engagement area to interlock with said loop-like concave rim and said protruding rim via gluing.
